(19) United States
(12) Patent Application Publication
(10) Pub. No.: US 2022/0183819 A1
(43) Pub. Date: Jun. 16, 2022

(54) INTRAOCULAR LENS COMPRISING AN ARM WITH AN ADJUSTABLE LENGTH AS A HAPTIC

(71) Applicant: Carl Zeiss Meditec AG, Jena (DE)

(72) Inventor: Stephanie Mueller, Neuruppin (DE)

(21) Appl. No.: 17/683,202

(22) Filed: Feb. 28, 2022

Related U.S. Application Data
(63) Continuation of application No. PCT/EP2020/072217, filed on Aug. 7, 2020.

(30) Foreign Application Priority Data
Aug. 30, 2019 (DE) 10 2019 123 300.3

(51) Int. Cl. A61F 2/16

(52) U.S. Cl. CPC A61F 2/16; A61F 2002/1683; A61F 2002/169053; A61F 2002/16901; A61F 2250/0018

(57) ABSTRACT

An intraocular lens is provided which includes an optical part and a haptic, which is coupled to the optical part, and with a main optical axis, which intersects a front side and a rear side of the optical part, wherein the haptic has at least one first haptic part, which is configured as a strand-like clip, wherein the strand-like clip has a longitudinal axis, wherein the strand-like clip has at least one length-changing apparatus with which the strand-like clip is variable in its length in a defined manner in the direction of its longitudinal axis.

### CROSS REFERENCE TO RELATED APPLICATIONS

This application is a continuation application of international patent application PCT/EP2020/072217, filed Aug. 7, 2020, designating the United States and claiming priority to German application 10 2019 123 300.3, filed Aug. 30, 2010, and the entire content of both applications is incorporated herein by reference.

### TECHNICAL FIELD

The disclosure relates to an intraocular lens with an optical part and with a haptic, which is coupled to the optical part, and with a main optical axis that intersects a front side and a back side of the optical part, wherein the haptic has a first haptic part, which is configured as a strand-like clip, wherein the clip has a longitudinal axis.

### BACKGROUND

Intraocular lenses are known in various embodiments. Typically, intraocular lenses have at least two separate haptics formed opposite one another in circumferential direction around the main optical axis and formed so as to radially adjoin the optical part. It is also possible for more than two such separate haptics to be formed, for example three haptics.

Intraocular lenses may be implanted in place of a natural lens of the eye at different defined positions within the eye. It is thus envisaged in this context that specific intraocular lenses are implanted in an anterior chamber of the eye. For example, such anterior chamber lenses may be fixed in the anterior iridocorneal angle.

Also known are intraocular lenses that are referred to as iris clip lenses. Such intraocular lenses are secured to the pupil. In particular, they are clipped to the pupil opening. Such an intraocular lens is known, for example, from DE 10 2007 057 122 A1. The intraocular lens therein, with this specific implantation site in the eye, has two opposite haptics. Each of these haptics has two L-shaped haptic arms. Mutually facing ends of these haptic arms are, viewed in a plane at right angles to the main optical axis of this intraocular lens, arranged so as to face one another, but arranged contactlessly and without overlap. Haptic arms formed in this way can clip onto the iris by way of the gap formed in circumferential direction around the main optical axis between the ends of the haptic arms. However, such lenses are not intended for and not suitable for implantation in a capsular bag of an eye.

In this regard, there are further known specific intraocular lenses, which can be referred to as posterior chamber lenses and can be implanted into a capsular bag of the eye.

DE 103 10 961 B4 discloses an intraocular lens, which is a posterior chamber lens. In this posterior chamber lens, two separate haptics are formed radially adjoining the optical part in opposite regions of the optical part. Both the respective haptics are formed with two haptic parts. The two haptic parts of a haptic are movable relative to one another. For this purpose, a defined kinked site, for example in the form of a film hinge, is formed at a defined connection site between the haptic parts that are connected to one another in one piece. In this way, the radially outer haptic part of this haptic can be kinked or folded relative to the first haptic part, which directly adjoins the optical part. This folding motion is possible only in the plane at right angles to the optical axis. This is intended to reduce the radial width of the intraocular lens as a whole in order to be able to avoid irritation in the interior of the capsular bag resulting from these haptics.

An intraocular lens is known from U.S. Pat. No. 4,077,071 B, which has simple clips as haptics. These longitudinally stiff clips are hollow and formed as tubes. Consequently, the problem that they can be arranged in capsular bags of different sizes only with limited positional stability also arises here. The intraocular lens may tilt and/or rotate in the capsular bag. This leads to disadvantages in terms of vision.

### SUMMARY

It is an object of the present disclosure to provide an intraocular lens which facilitates improved positioning in a capsular bag of an eye.

This object is achieved by an artificial intraocular lens as described herein.

An aspect of the disclosure relates to an artificial intraocular lens with an optical part. The optical part is a lens. The optical part has specific optical imaging properties such that specific correction of visual defects can be realized therewith.

Moreover, the intraocular lens includes a haptic which is coupled to the optical part. The intraocular lens has an optical axis or a main optical axis, which intersects a front side and a back side of the optical part and in particular intersects these centrally in the middle region.

The haptic has at least one first haptic part. The first haptic part is designed as a strand-like clip. This clip has a longitudinal axis. The clip has at least one length-changing apparatus with which the clip itself is variable in terms of its length in a defined manner in the direction of its longitudinal axis. This configuration thus creates a clip which is variable in itself in terms of its length in a defined manner. This length is also variable in a specific direction, to be precise viewed in the direction of its longitudinal axis. With such a configuration, the length of such a strand-like clip can be set individually. This results in lengths of a clip that differ as defined depending on the situation and thus lead to intraocular lenses of different sizes. Such a variable length setting of a specific part of the intraocular lens, to be precise of this strand-like clip, allows the intraocular lens to be implanted in a stable position in capsular bags of different sizes. It is therefore no longer necessary to provide a wide range of different separate intraocular lenses for different capsular bags. Rather, it is now possible to use a single type of intraocular lens to produce an adapted implantation size that can be adapted to an individual size of a capsular bag by adapting the length of the haptic accordingly. As a result, both undesired tilting and undesired rotation of the intraocular lens in a capsular bag are prevented in an improved manner.

In an exemplary embodiment, the length-changing apparatus has at least one telescopic connection. A telescopic connection is understood to mean a configuration in which two separate partial elements of the clip are guided within one another and can move, in particular slide, relative to one another in the direction of the longitudinal axis of the clip. As a result, different defined lengths of the clip can be set when the two partial elements are in a coupled state in which they are guided within one another. In particular, such a length-changing apparatus is configured as a telescopic connection. The length-changing apparatus can also have at least two separate telescopic connections that are formed in the clip.

A telescopic connection is a mechanically stable configuration that maintains the mechanical strength of the haptic as such. A very directed change in length is also made possible by this state in which two separate partial elements are guided within one another. This prevents undesirable tilting of the partial elements relative to one another when the length is changed. Last but not least, such a telescopic connection also keeps a desired, defined length setting stable. A telescopic connection also forms a mechanically robust and resilient interface, especially in the overlap region of the partial elements that are guided within one another.

Typically, the length-changing apparatus has at least one accordion-like folding part. This accordion-like folding part is elastically variable in length in the direction of the longitudinal axis of the clip. Such an embodiment allows, in particular, discrete steps of the change in length. In the case of an accordion-like folding part, a plurality of such corrugated structures are typically formed, which can be pulled apart or pushed together in the direction of the longitudinal axis. In this way, this change in length can be set. A configuration with an accordion-like folding part also allows the length-changing apparatus to be formed in one piece. This can save manufacturing costs and assembly costs. In this context, positional tolerances that can occur over time between a plurality of partial elements of a length-changing apparatus can also be avoided in particular.

In an exemplary embodiment, the clip is formed at least partly as a tube. Such a hollow line makes it possible to reduce the weight of the haptic. In addition, this configuration also creates the installation space to be able to realize a telescopic connection and/or to produce an accordion-like folding part and also to functionally actuate it accordingly. Moreover, such a configuration as a hollow tube also provides the desired deformation elasticity of the clip at least in partial regions.

In an exemplary embodiment, the haptic has a second haptic part, which is formed separately from the first haptic part. This second haptic part is functionally configured corresponding to the first haptic part with at least one length-changing apparatus. Such a configuration allows the better implantation of the intraocular lens in a capsular bag in a stable position. Basic retention of the intraocular lens in the capsular bag is increased by at least two separate haptics. Since both haptic parts are then configured with at least one length-changing apparatus, the size of the intraocular lens can be set particularly finely as needed by adjusting the lengths of the first haptic part and/or the second haptic part. This allows very precise need-based adaptation of the sizes of the haptics to the circumstances of the capsular bag in which the intraocular lens is to be implanted. This is true both with regard to the length and, if appropriate, the shape.

The two haptic parts can be connected to one another. In this context, the stability of the haptic can be increased. In particular, the two haptic parts connected to one another form a circumferential haptic ring. This haptic ring is thus completely closed and therefore formed without interruption. In such a configuration, the haptic ring forms, as it were, a frame around the optical part.

In particular, a longitudinal axis of a haptic part extends in one plane viewed over the entire length. In particular, this plane is oriented perpendicular to the main optical axis. Typically, the longitudinal axes of two haptic parts, if at least such a number of haptic parts is formed, can each extend over their entire length in one plane, in particular a common plane.

In one exemplary embodiment, the haptic can be arranged directly at the optical part, in particular be attached to it.

The intraocular lens can be formed in one piece. In such a configuration, the haptic is then also formed in one piece with the optical part, in particular from a polymer material.

However, the intraocular lens can also have a multi-part design. For example, the optical part can here constitute a first partial component of the intraocular lens, and the at least one haptic part can constitute a separate, second partial component of the intraocular lens. These two separate partial components can then be connected in different ways. For example, a mechanical connection, such as a plug-in connection or a latch connection or the like, can be provided here.

In an exemplary embodiment, the strand-like clip can have an opening on a side facing the optical part. An edge of the optical part can engage in this opening such that the optical part is held in the haptic part. Such a configuration, in which the partial components of the intraocular lens are separate parts, allows a simple mechanical connection of the partial components, which nevertheless makes high and permanent stable retention possible.

The opening is formed in particular as a slot. The opening extends in particular in the direction of the longitudinal axis of the clip.

In particular in such a configuration, the optical part may have a coupling web on the radially outer edge region. This coupling web is then intended to engage in this opening. The coupling web can be configured as a bead or rail. In particular, this coupling web is an additional integrated region of the optical part, but has no optical imaging properties. In particular, it is only provided for mechanically coupling to the haptic. The result of this is that the basic configuration of the optical part is not restricted or is not impaired with regard to the region which provides the optical imaging property. In particular, this then also avoids the situation in which the region of the optical part which is responsible for the optical imaging property does not dip into the opening. As a result, a reduction in the optically imaging regions of the optical part can be avoided and, in addition, damage to this optically imaging region of the optical part or rubbing or the like against the opening of the clip can be avoided.

Such a coupling web can be formed in a straight line. It then extends around the main optical axis only by a small partial length of the entire circumferential length of the outer radial edge of the optical part. However, this coupling web can also be curved. For example, it can constitute a radially outer ring of the optical part, which can be formed to be at least partially circumferential, in particular also completely circumferential. This allows the azimuthal position of the optical part relative to the haptic to be set individually. This is advantageous in particular if the optical part is also designed to correct astigmatism, for example. This is because an individual position of the optical part relative to the haptic can be set, with the result that the haptic can be optimally positioned in the capsular bag and, in addition, the correction of this visual defect, in particular the astigmatism, can be corrected for the individual eye.

In an advantageous embodiment, the length-changing apparatus is designed to be variable in length in discrete steps. In this context, the changes in length can occur discretely, for example in millimeter increments or in half-millimeter increments or, however, also in value increments larger than one millimeter. At least two such discrete length change steps may be realized. However, more than two, for example more than five or for example more than ten, may also be realized.

In an alternative exemplary embodiment, a length-changing apparatus may be continuously adjustable in length. As a result, an even more finely adjusted setting can be accomplished.

A length-changing apparatus may extend over only a partial length of the total length of the clip. For example, this partial length can be less than half the overall length of the clip. However, the partial length can also be less than a third, in particular less than a quarter, of the entire length of a clip.

A length-changing apparatus may be formed in a length region of the clip that constitutes an end piece of said clip. In such an exemplary embodiment, the length-changing apparatus thus constitutes an end termination of the clip viewed along a longitudinal axis. The length-changing apparatus may be formed locally at the site in the clip at which the clip is mechanically connected, in particular directly connected, to the optical part. A length-changing apparatus can thus be formed in a longitudinal section of the clip that is arranged closer to the main optical axis in the radial direction than a longitudinal section that is radially further away from it. In particular, the longitudinal section of the clip that is radially closer to the main optical axis can be an end piece of the clip.

In particular, if such a clip has a U-shape, a length-changing apparatus can be the free end of a U-leg.

In particular, in such a configuration, the respective ends of the U-legs can be configured as a length-changing apparatus. In this context, these then at least two length-changing apparatuses are identical in terms of design and thus functionality. For example, two telescopic connections can be formed in this context. However, it is also possible for two accordion-like folding parts to be formed.

It is also possible that, as a further concrete configuration of a length-changing apparatus, not only in this configuration, spring elements are configured as the length-changing apparatuses. In this context, a spring element can be, for example, a cylindrical spring that is elastically deformable in the direction of the longitudinal axis. This can be made from a plastics material, for example. A spring element can also be surrounded by another material. For example, it can be completely surrounded. In this context, the spring element may also be surrounded by a polymer material. Such a spring element may also be overmolded by a material.

A further exemplary embodiment of a spring element can, however, also be a cylindrical, solid molded body which is elastically deformable in the direction of its longitudinal axis, which corresponds to the longitudinal axis of the clip. Correspondingly deformable plastics materials may be mentioned here, for example.

In such a configuration, such a cylindrical molded body can also be made from a porous material. As a result, the weight can be reduced and, if necessary, in particular the deformation elasticity can be increased.

In an alternative exemplary embodiment, if the haptic has at least two separate length-changing apparatuses, the latter are of different design and thus different functionality. Possible combinations are possible in this context, for example, by way of a telescopic connection with an accordion-like folding part or a spring element. Combinations of an accordion-like folding part and a spring element are also possible.

In exemplary embodiments in which a change in length is made possible in discrete length steps, the length-changing apparatus can have latching steps in this regard. As a result, the set length steps can also be maintained.

The length-changing apparatus can also have two haptic parts, of which each haptic part is formed from two separate partial elements, which are coupled to one another in a length-variable manner in the direction of the longitudinal axis. For example, two at least partially hollow tubes can be provided here for each haptic part, which are guided within one another and are correspondingly mechanically coupled in order to be able to set a defined change in length of a respective haptic part. It is also possible for only one of the two partial elements to be partially hollow in order to be able to insert the other partial element.

In the finished final state of an intraocular lens, the optical part may also be arranged in the circumferential direction around the main optical axis completely without contact with the haptic. In such a configuration, a connecting element, such as a piece of wire, can be formed between the optical part and the haptic, in particular the strand-like clip. This achieves a specific distance between the optical part and the haptic, while still enabling the stable mechanical connection between the haptic and the optical part that is separate therefrom.

A strand-like clip, which constitutes a haptic part, can also have at least two separate length-changing apparatuses arranged spaced apart from one another in the direction of the longitudinal axis. For example, these two length-changing apparatuses can each be formed in the clip itself spaced apart from the ends of the clip, which is in particular U-shaped. However, a length-changing apparatus can also be formed as an end piece of such a clip and the second length-changing apparatus can be arranged spaced apart from it in the direction of the longitudinal axis of the clip. For example, in relation to the overall length of the clip, this further length-changing apparatus can be formed approximately in the middle region of the clip.

A symmetrical structure can likewise be provided, in which two length-changing apparatuses each form the end pieces of the in particular U-shaped clip, and a further third length-changing apparatus is formed in the clip, viewed in the direction of the longitudinal axis of the clip, to be spaced apart therefrom, in particular approximately centrally in relation to the overall length of the clip.

In the exemplary embodiments in which at least two separate length-changing apparatuses are formed in a clip, these length-changing apparatuses may also differ with regard to the length setting. This means that one length-changing apparatus is configured, for example, for discretely changing the length in length steps, and the further length-changing apparatus is configured for continuous length adjustment.

A haptic part may also have two separate partial elements which, viewed individually, are each formed in the manner of a strand. Each partial element may also be connected with an end region to the optical part, in particular at its perimeter. The respectively other end of these partial elements, which projects freely in the non-coupled state, is then configured in particular in such a way that they can be guided within one another. As a result, a length-changing apparatus, in particular a telescopic connection, that is formed spaced apart from the connection sites on the optical part can be configured in a simple manner. In this context, the intraocular lens can in particular be manufactured in one piece, in particular from a polymer material, and subsequently said freely projecting ends of the partial elements of the haptic part can be guided within one another and thus be slidably coupled. This creates the uninterrupted, in particular U-shaped, strand-like clip, which has an integrated length-changing apparatus, in particular as a telescopic connection.

This telescopic connection can also have latching steps in order to form a telescopic connection not for continuous adjustment but for discrete adjustment of the length of the clip.

In an exemplary embodiment, a length-changing apparatus is integrated into the clip. In particular, it is thus formed in one piece with the remaining part of the clip.

In particular, the intraocular lens is configured as an intraocular lens implanted in a capsular bag. It may also be referred to as a capsular bag-implanted intraocular lens in this context. In particular, it is a posterior chamber lens for implantation into a capsular bag of an eye in this context. This means that the intraocular lens is intended, in particular solely, for implantation into a capsular bag of an eye.

Further features of the disclosure are apparent from the claims, the figures, and the description of the figures. The features and combinations of features mentioned in the description above and the features and combinations of features mentioned in the description of the figures below and/or shown only in the figures may be used not only in the respectively specified combination but also in other combinations, without departing from the scope of the disclosure. The disclosure shall thus also be considered to include and disclose embodiments of the disclosure that are not shown and elucidated explicitly in the figures, but emerge and can be created from separate combinations of features from the details elucidated. Disclosure shall also be considered to extend to embodiments and combinations of features that thus do not have all the features of an independent claim as originally worded. Disclosure shall additionally be considered to extend to embodiments and combinations of features, in particular by virtue of the embodiments explained above, which go beyond or depart from the combinations of features set out in the dependency references of the claims.

The specific values indicated in the documents for parameters and indications concerning ratios of parameters or parameter values for the definition of exemplary embodiments of the eye lens should be considered to be concomitantly encompassed by the scope of the disclosure even in the context of deviations, for example on account of measurement errors, system faults, DIN tolerances, etc., which is also understood to mean explanations relating to substantially corresponding values and indications.

### BRIEF DESCRIPTION OF THE DRAWINGS

The disclosure will now be described with reference to the drawings wherein:

FIG. 1 shows a simplified illustration of an exemplary embodiment of an intraocular lens according to an exemplary embodiment of the disclosure, which has already been implanted in particular in a capsular bag;

FIG. 2 shows a simplified illustration of a further exemplary embodiment of an intraocular lens according to the disclosure, which has already been implanted in particular in a capsular bag;

FIG. 3 shows an illustration in accordance with FIG. 1 and FIG. 2 with a third exemplary embodiment of an intraocular lens according to the disclosure;

FIG. 4 shows an illustration in accordance with FIG. 1 to FIG. 3 with a fourth exemplary embodiment of an intraocular lens according to the disclosure;

FIG. 5 shows a top view and a side view of an optical part of an intraocular lens with specific coupling webs for coupling to a haptic of the intraocular lens;

FIG. 6 shows an illustration according to FIG. 5 with designs of coupling webs differing therefrom;

FIG. 7 shows a top view of an exemplary embodiment of an intraocular lens, in which the haptic parts are shown in a basic state with their lengths; and

FIG. 8 shows an illustration of the embodiment of the intraocular lens according to FIG. 7, in which the haptic parts are increased in terms of length.

### DESCRIPTION OF EXEMPLARY EMBODIMENTS

In the figures, identical or functionally identical elements are given the same reference signs.

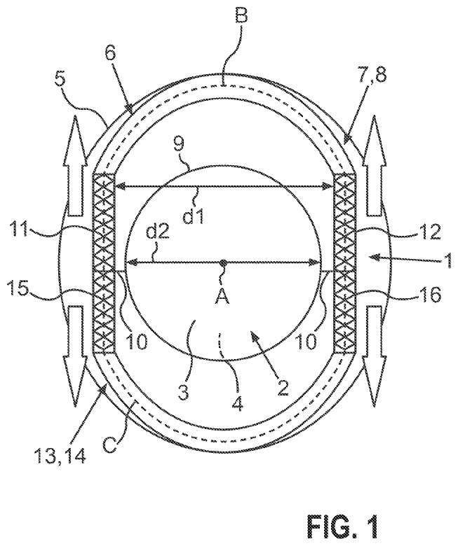

FIG. 1 shows a perspective illustration of an exemplary embodiment of an artificial intraocular lens 1. This intraocular lens 1 is a posterior chamber lens for implantation into a capsular bag of an eye. It may therefore also be referred to as a capsular bag-implanted intraocular lens. The intraocular lens 1 includes an optical part 2. The optical part 2 is in the form of a lens. It is configured to create a defined optical imaging characteristic of the intraocular lens 1. The intraocular lens 1 has an optical axis or a main optical axis A. The latter passes through a front side 3 of the optical part 2 and a back side 4 of the optical part 2, centrally and in the middle region of the optical part 2. The main optical axis A is oriented perpendicular to the plane of the figure.

The intraocular lens 1, which is shown as an example implanted in a capsular bag 5 in the illustration in accordance with FIG. 1, is held in a stable position therein. For this purpose, the intraocular lens 1 has a haptic 6 which is specifically configured. In the exemplary embodiment shown in FIG. 1, the haptic 6 has a first haptic part 7. This first haptic part 7 is configured as a strand-like clip 8. This clip 8 has a longitudinal axis B. This clip 8 is formed in the exemplary embodiment with a U-shape. The clip 8 is curved in a partial region, wherein this partial region has a maximum arc width d1, which is larger than a diameter d2 of the optical part 2. The first haptic part 7 and thus the strand-like clip 8 can be arranged directly at the optical part 2, in particular at a circumferential edge 9. However, as is provided in the illustration in FIG. 1, this clip 8 can also be arranged radially spaced apart from the optical part 2 and thus also from the circumferential edge 9. Such a configuration can be provided in particular when the optical part 2 and the haptic 6 are separate components. A connecting element 10 can be provided for connecting the haptic 6 to the optical part 2, in particular with such a spaced-apart positioning. This connecting element 10 can be a wire, for example. For this purpose, a notch or a groove may be formed in the circumferential edge 9, in which the wire 10 is placed and then guided radially projecting outward to the side in order to be connected to the haptic 6.

The clip 8, which in the exemplary embodiment surrounds the optical part 2 over an azimuth length of in particular 180°, has at least one length-changing apparatus 11. In the exemplary embodiment, the clip 8 has two length-changing apparatuses 11 and 12. In the exemplary embodiment, the length-changing apparatuses 11 and 12 are formed as end pieces of this U-shaped, strand-like clip 8. The length-changing apparatuses 11 and 12 are formed in such a way that their length is variable in a defined manner in the direction of the longitudinal axis B. As a result, the length of the clip 8 is also variable in a defined manner viewed in the direction of its longitudinal axis B. In the exemplary embodiment shown in FIG. 1, the haptic 6 typically has a further, second haptic part 13. The latter is advantageously configured to correspond to the first haptic part 7. Like the first haptic part 7, it can be hollow in design at least in part. For example, a hollow tube can be provided here.

In the exemplary embodiment shown, the second haptic part 13 is likewise formed as a strand-like clip 14. This strand-like clip 14 has a longitudinal axis C. In particular, the strand-like clip 14 has at least one length-changing apparatus 15. In particular, in the exemplary embodiment shown, it likewise has two separate length-changing apparatuses 15 and 16, which are also arranged spaced apart from one another. In an exemplary embodiment, these are designed as end pieces of the U-shaped clip 14.

As can be seen in the illustration in FIG. 1, in one exemplary embodiment, the two clips 8 and 14 are connected directly to one another at their respective distal ends. As a result, they form a circumferentially closed clip, which constitutes the haptic 6. The optical part 2 is thus surrounded by a circumferentially closed, strand-like overall clip, which constitutes a haptic ring. In particular, the clips 8 and 14 each extend over their entire length along the longitudinal axis B, C viewed in a plane which is oriented in particular perpendicular to the main optical axis A.

A length-changing apparatus 11, 12, 15, 16 can be configured as a telescopic connection. However, a length-changing apparatus 11, 12, 15, 16 can also be configured as an accordion-like folding part, in particular comparable to a leporello. It is also possible for a length-changing apparatus 11, 12, 15, 16 to be configured as a spring element. In FIG. 1, all length-changing apparatuses 11, 12, 15, 16 are of the same design and have the same functionality. In particular, the length-changing apparatuses 11, 12, 15, 16 are configured as accordion-like folding parts. Due to their adjustability, which can be continuous or discrete, the length of a clip 8 and/or 14 can be increased or decreased in the direction of the respective longitudinal axis B and/or C. This is indicated by the symbolic arrows in FIG. 1.

The haptic 6 can also be connected directly to the optical part 2, in particular its circumferential edge 9. For example, the clips 8 and/or 14 can therefore have slots on the side facing the optical part 2. The optical part 2 can extend into said slots and be held therein. A one-piece configuration with the intraocular lens 1 is also possible. In the exemplary embodiment shown in FIG. 1, the length-changing apparatuses 11, 12, 15 and 16 are formed as end pieces of the respective clips 8, 14. It is also possible that at least one length-changing apparatus 11, 12, 15, 16 is not formed as such an end piece, but instead is formed in the middle region of the overall length of a clip 8, 14, for example. As can be seen, the length of a length-changing apparatus 11, 12, 15, 16 viewed in the direction of the longitudinal axis B or C is at most one third, in particular at most one quarter, of a respective overall length of a clip 8, 14.

The clips 8 and 14 can also be formed in one piece with one another. As a result, a clip ring is formed, which is designed to be fully circumferential.

In particular, a length-changing apparatus 11, 12, 15, 16 is integrated into a clip 8, 14, and is therefore in particular formed in one piece therewith.

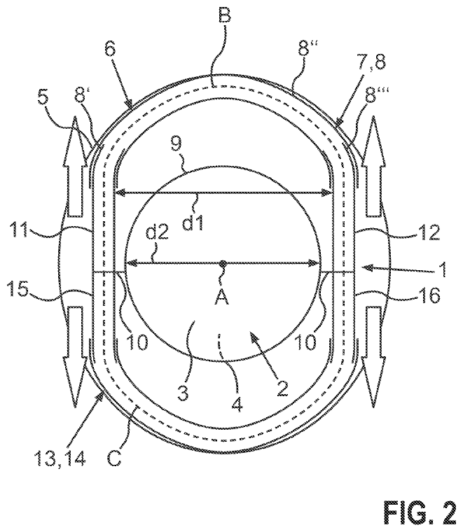

An intraocular lens 1 is shown in FIG. 2 in an illustration corresponding to that in FIG. 1. In contrast to the latter, the length-changing apparatuses 11, 12, 15, 16 are not configured as accordion-like folding parts, but as telescopic connections. The haptic part 7 with the strand-like clip 8 is formed here from at least two partial elements. For example, three partial elements can also be provided. These are a first partial element 8′, a second partial element 8″, and a third partial element 8′″. These three partial elements 8′, 8″, 8′″ are at least partially guided within one another and can move relative to one another in the direction of the longitudinal axis B in the state in which they are guided within one another. The further strand-like clip 14 can be constructed accordingly. The clip 8 can also be formed from only two partial elements which are movable relative to one another and are guided within one another. The same can be provided for the clip 14. In such an exemplary embodiment, the entire haptic 6 is then formed by four separate partial elements, in particular four tubes.

The further explanations relating to the arrangement and connection of the optical part 2 with the haptic 6 are possible according to FIG. 1. In the exemplary embodiment shown in FIG. 2, the number of length-changing apparatuses 11, 12, 15 and 16 is also to be understood merely as an example, so that more or fewer than these four mentioned can be provided here as well. The respective positions of these length-changing apparatuses 11, 12, 15, 16 are also to be understood as an example.

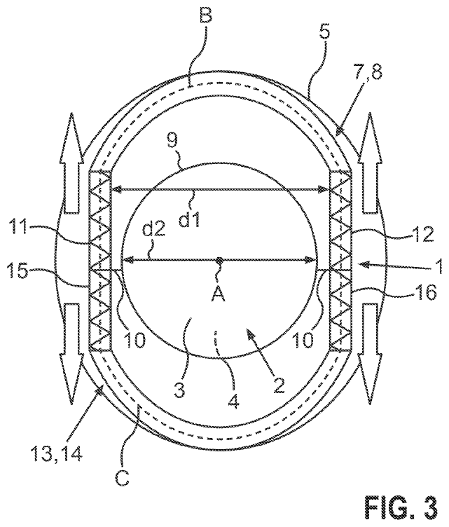

A further exemplary embodiment of an intraocular lens 1 is shown in FIG. 3 in an illustration corresponding to FIG. 1 and FIG. 2. In contrast to the illustration in FIG. 1 and FIG. 2, four length-changing apparatuses 11, 12, 15 and 16 are provided here, likewise by way of example, which are spring elements here. For example, axially resilient cylinder springs or axially deformable sleeve elements can be provided here. Here, too, the number and location of the length-changing apparatuses 11, 12, 15, 16 are to be understood as examples. Here, too, the alternative possible configurations, as explained in relation to FIG. 1, apply correspondingly.

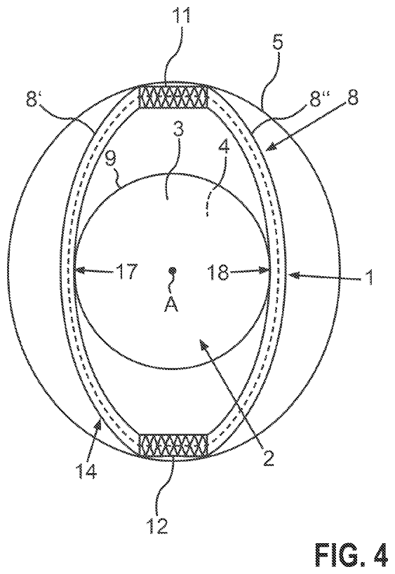

A further exemplary embodiment of an intraocular lens 1 is shown in FIG. 4. Said lens is also shown as an example already in the implanted state in a capsular bag 5. This schematic illustration shows that the haptic 6 is connected directly to the optical part 2, in particular at the circumferential edge 9. In this exemplary embodiment, an example is shown in which, in particular, a one-piece design of the intraocular lens 1 can be provided. In particular, length-changing apparatuses 11 and 12 are formed here in the strand-like clips 8 and 14 at a distance from the coupling sites with the circumferential edge 9. In particular, these length-changing apparatuses 11 and 12 are not provided as end pieces of the U-shaped clips 8 and 14 either. With regard to the U-shape of the clips 8, 14, the length-changing apparatuses 11, 12 are formed approximately in the middle region of the respective overall length of the clips 8 and 14. The clips 8 and 14 can also be formed in one piece and are therefore formed as a one-piece haptic ring that is circumferentially closed.

In particular, at least one length-changing apparatus 11, 12 is configured as a telescopic connection. For this purpose, said clip 8 has two partial elements 8′ and 8″, which are connected at one end to the optical part 2 at the connection sites 17 and 18, which are embodied in particular offset from one another by 180° in the circumferential direction about the main optical axis A, to the circumferential edge 9. The other ends of these partial elements 8′ and 8″ are guided in this respect within one another at a distance from the optical part 2, and therefore a telescopic connection is configured in this respect as a length-changing apparatus 11. A length-changing apparatus 11 and/or 12 can also be configured in this example as an accordion-like folding part or as a spring element.

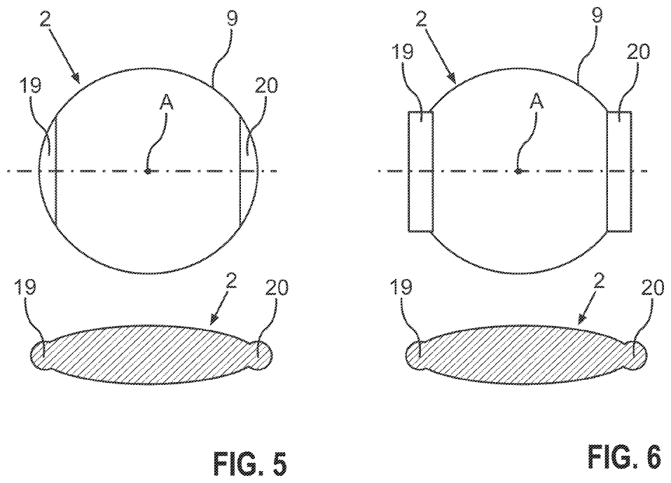

FIG. 5 shows a simplified illustration of an optical part 2 in a top view. It shows that coupling webs 19 and 20 are formed on opposite sides. These coupling webs 19 and 20, which can likewise be seen in the lower sectional illustration of the optical part 2, serve to be received in openings already mentioned above, in particular slots, in the clips 8 and/or 14. As a result, the separate optical part 2 can be fastened to the haptic 6 in a stable position. The sectional illustration in FIG. 5 is to be understood to be merely an example. The sectional surfaces of the coupling webs 19 and 20 are only intended to illustrate the respective geometry. In particular, the optical part 2 is formed in one piece with the coupling webs 19 and 20. The coupling webs 19, 20 are shaped on the outside here in such a way that the constant circular shape of the circumferential edge 9 is not changed.

A further exemplary embodiment of an optical part 2 with coupling webs 19 and 20 is shown in FIG. 6 in an illustration corresponding to that in FIG. 5. In contrast to the illustration according to FIG. 5, a straight coupling web 19 and 20 is formed in a top view in the exemplary embodiment shown in FIG. 6, which does not adopt the curvature of the circumferential edge 9 at its ends. The lower schematic sectional illustration in FIG. 6 again shows a sectional illustration along the dashed line of the top view in FIG. 6.

In the example of coupling webs 19 and 20 shown here, the latter are partially configured to circumferentially extend around the main optical axis A. Exemplary embodiments in which such coupling webs are configured as one coupling web which is configured to be fully circumferential can also be provided.

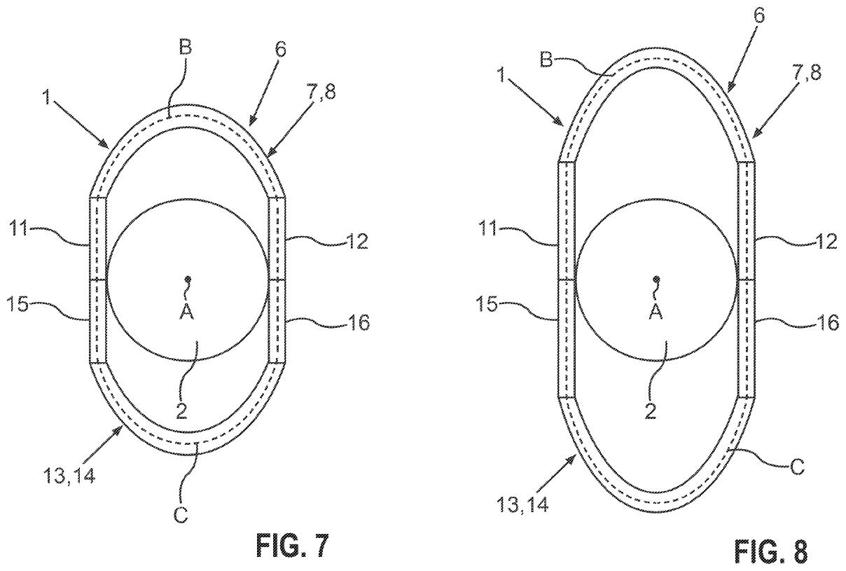

FIG. 7 shows a top view of an exemplary embodiment of an intraocular lens 1. The haptic parts 7 and/or 8 are set here in a basic state with respect to their lengths. This means that the length-changing apparatuses 11 and/or 12 and/or 15 and/or 16 do not have increased lengths and are therefore not extended.

In contrast, FIG. 8 shows schematically the situation of the intraocular lens according to FIG. 7, in which at least one, in particular all length-changing apparatuses 11, 12, 15, 16 have increased lengths. An increase in length that is the same for all length-changing apparatuses 11, 12, 15, 16 can be provided not only in this example, but also generally. However, the changes in length of the length-changing apparatuses 11, 12, 15, 16 can also differ.

In general, a length-changing apparatus 11 and/or 12 and/or 15 and/or 16 can be flexurally stiff or flexible, in particular elastically. Long, stiff and straight sections of a haptic part can thus be avoided, particularly in the extended state. In particular, by bending, an arc shape can also be continued in the region of a length-changing apparatus.

It is understood that the foregoing description is that of the exemplary embodiments of the disclosure and that various changes and modifications may be made thereto without departing from the spirit and scope of the disclosure as defined in the appended claims.

What is claimed is:

1. An intraocular lens, comprising:
an optical part having a front side and a back side; and
a haptic coupled to the optical part,
wherein the intraocular lens defines a main optical axis, which intersects the front side and the back side of the optical part,
wherein the haptic has at least one first haptic part configured as a strand-like clip,
wherein the strand-like clip defines a longitudinal axis,
wherein the strand-like clip has at least one length-changing apparatus with which the strand-like clip is variable in terms of its length in a defined manner in a direction of the longitudinal axis, and
wherein the strand-like clip defines an opening on a side facing the optical part, into which opening the radially outer edge of the optical part engages such that the optical part is held in the at least one first haptic part.

2. The intraocular lens as claimed in claim 1, wherein the opening is a slot.

3. The intraocular lens as claimed in claim 1, wherein the at least one length-changing apparatus has at least one telescopic connection which changes elastically in length in the direction of the longitudinal axis.

4. The intraocular lens as claimed in claim 1, wherein the at least one length-changing apparatus has at least one accordion-like folding part which changes elastically in length in the direction of the longitudinal axis.

5. The intraocular lens as claimed in claim 1, wherein the strand-like clip is at least partially formed as a tube.

6. The intraocular lens as claimed in claim 1, wherein the haptic has a second haptic part configured with a second length-changing apparatus functionally corresponding to the at least one first haptic part.

7. The intraocular lens as claimed in claim 6, wherein the first and second haptic parts are connected to one another and form a circumferential haptic ring.

8. The intraocular lens as claimed in claim 1, wherein the at least one length-changing apparatus is configured to be variable in length in discrete steps.

9. The intraocular lens as claimed in claim 1, wherein the at least one length-changing apparatus is integrated into the strand-like clip.

10. The intraocular lens as claimed in claim 1, wherein the intraocular lens is a posterior chamber lens for implantation into a capsular bag of an eye.
